(19) United States
(12) Patent Application Publication
(10) Pub. No.: US 2011/0104311 A1
(43) Pub. Date: May 5, 2011

(54) Performance Beverage

(76) Inventor: John Ronald Dooley, Madison, MS (US)

(21) Appl. No.: 12/917,450

(22) Filed: Nov. 1, 2010

Related U.S. Application Data
(60) Provisional application No. 61/256,734, filed on Oct. 30, 2009.

(51) Int. Cl. A61K 36/00; A61P 43/00

(52) U.S. Cl. 424/725

(57) ABSTRACT

A performance beverage for underwater diving or other challenging physiological environments.

### PRIORITY

This nonprovisional patent application claims the benefit of U.S. provisional patent application No. 61/256,734 filed on Oct. 30, 2009.

### FIELD OF THE DISCLOSURE

The field of the disclosure is performance beverages.

### BACKGROUND

Underwater divers face a hostile environment because of physiological reasons, among others: dehydration, high pressure, decompression sickness, cramping, metabolic demands from high levels of muscle exertion, oxidative stress from production of reactive oxygen species (ROS) and from lack of oxygen for needy tissues, and the artificial oxygen supply itself. Although other performance beverages exist, none addresses the unique challenges created by diving. Thus, there is a great need for a performance beverage which helps divers address the physiological needs of the diving environment and keep their bodies damage free. Such a beverage would also be beneficial to persons in other environments such as space travel, or any other situation involving high exertion or high pressure conditions.

### SUMMARY OF THE INVENTION

The present invention is a powder performance beverage mix which provides performance enhancing nutrients to protect divers from dehydration, high pressure, decompression sickness, cramping, metabolic demands from high levels of muscle exertion, oxidative stress from production of ROS and from lack of oxygen for needy tissues, and the artificial oxygen supply itself. The invention thus improves performance in and recovery from diving. The present invention boosts energy and helps to hydrate the body. L-arginine increases Nitric Oxide (NO) availability which reduces gas bubble formation. L-arginine also increases levels of blood glucose and blood insulin to boost the body's available energy from its own stores, and also decreases free fatty acids in the blood. L-arginine also improves nitrogenous waste removal and provides substrate for the urea cycle and assists in the Citric Acid Cycle (or Krebs Cycle) for production of energy. N-acetyl-Cysteine increases levels of glutathione, a vitally important anti-oxidant in the body, and maintains mucus membrane integrity. The ionic electrolytes potassium, calcium, magnesium, zinc, copper, and manganese add to hydration and create hyperosmotic conditions in blood vessels which is one tool for preventing gas bubble formation in blood vessels. Curcumin provides anti-oxidation and boosts heme-oxygenase 1 in endothelial tissues which, especially in the respiratory system, are under stress in diving on account of high pressure and dry oxygen from tanks. Noni extract also provides anti-oxidants. Additional anti-oxidants, vitamin C and E, work to protect divers from high levels of Alanine Aminotransferase (ALT) and AST Aspartate Aminotransferase which are liver enzymes resulting from high superoxide formations.

### DETAILED DESCRIPTION

The invention is blended to maximize performance and also to have a pleasant taste. In a preferred embodiment of 1 Liter aqueous solution, the invention's ingredients comprise, in the following approximate amounts:

| | | Amt Per | |
|---|---|---|---|
| Ingredient | Other Name | Serving | |
| Calcium | Calcium (ionic) | 250 | mg |
| Magnesium | | | |
| Lactate | | | |
| Magnesium | Magnesium (ionic) | 250 | mg |
| Sulfate | | | |
| Dipotassium | Potassium (ionic) | 450 | mg |
| Phosphate | | | |
| Zinc Sulfate | Zinc (ionic) | 3 | mg |
| Copper Sulfate | Copper (ionic) | 3 | mg |
| Manganese | Manganese (ionic) | 1 | mg |
| Sulfate | | | |
| Vitamin A | Vitamin A | 200 | IU |
| Acetate | | | |
| d-Calcium | Vitamin B5 - Pantothenic Acid | 500 | mg |
| Pantothenate | | | |
| Ascorbic Acid | Vitamin C | 250 | mg |
| Cyanocobalamin | Vitamin B12 | 1000 | mg |
| Pyrodoxine HCL | Vitamin B6 | 50 | mg |
| Fumaric Acid | | 50 | mg |
| Citric Acid | | 25 | mg |
| Folic Acid | Vitamin B9 | 50 | mcg |
| N-Acetyl-L- | N-Acetyl-Cysteine | 500 | mg |
| Cysteine | | | |
| L-Arginine | | 250 | mg |
| Curcumin | Curcuma Longa. Extract of Turmeric | 150 | mg |
| Meriva ® | Root (Formulation including ratio | | |
| | of components in 1:2:2 as follows: | | |
| | 30 mg Curcumin, 60 mg Soy | | |
| | Phosphatidylcholine, and 60 mg | | |
| | Microcrystalline cellulose) | | |
| Noni Extract | Morinda Citrofolia L. | 975 | mg |
| | | (including 5 | |
| | | mg Sodium) | |
| Biotin | Vitamin B7 | 100 | mcg |
| Inositol | | 50 | mg |
| Natural Flavor | | | |
| Stevia (Reb A), | | | |
| Sucralose | | | |

The reader will appreciate that the exact amounts of the ingredients may be varied in other embodiments in accordance with the present disclosure. Further, in other embodiments certain ingredients not listed above may be added, and some listed could be deleted, without changing the utility of the invention. The invention can exist either in aqueous solution or in a dry powder mix which is later dissolved in water.

Noni. The noni fruit is known to have anti-oxidant properties and to influence hemoglobin positively. Yang, et al., Free-radical-scavenging activity and total phenols of noni (Morinda citriofolia L.) juice and powder in processing and storage, Food Chemistry 102: 302-308 (2007); Palu, et al., Morinda citrifolia L. (noni) improves athlete endurance: its mechanisms of action, J. Medicinal Plants Res. 2(7): 154-58 (2008). Divers face mental stress in underwater environmental conditions from relative weightlessness, vertigo, and disorientation but noni has been shown to protect brains from stress-induced impairment of cognitive function and this protection may result from improvement of stress-induced decreases in blood vessel density in the hippocampal dentate gyrus. Muto, et al., Morinda citrifolia fruit reduces stress-induced impairment of cognitive function accompanied by vasculature improvement in mice, Physiology & Behavior 101:211-217 (2010). Noni has also been shown to protect against brain injury after ischemic events or a model of degenerative disease. Harada, et al., Preventive effect of morinda citrifolia fruit juice on neuronal damage induced by focal ischemia, Biol. Pharm. Bull. 23(3): 405-09 (2009); Muralidharan, et al., Protective effect of morinda citrifolia fruits on β-amyloid (25-35) induced cognitive dysfunction in mice: an experimental and biochemical study, Phytother. Res. 24:252-58 (2010). A preferred embodiment of the invention contains approximately 975 mg of noni extract for dissolving in 1 liter of water, but a range of approximately 500-5000 mg per liter would also be effective in the invention. The powder extract of the fruit noni is of the kind and quality obtained from Healing Noni in Hilo, Hi.

Curcumin. During and after decompression from dives, gas bubbles are formed from excess gas taken into tissues from compressed gases consumed, and these bubbles can lead to endothelial dysfunction in the lungs as well as in the arteries. Brubakk, et al., A single air dive reduces arterial endothelial function in man, J. Physiol. 566.3, pp. 901-06 (2005). The rhizome of Curcuma Longa (also known as turmeric root) has a chemical formula of C₂₁H₂₀O₆. Curcumin has been shown to increase expression of the cytoprotective compound heme-oxigenase-1 in endothelial cell cultures. Motterlini, et al., Curcumin, an anti-oxidant and anti-inflammatory agent, induces heme oxygenase-1 and protects endothelial cells against oxidative stress, Free Radic. Biol. Med. 2000; 28:1303-12; Gil-Saeng Jeong, et al. Comparative effects of curcuminoids on endothelial heme oxygenase-1 expression: ortho-methoxy groups are essential to enhance heme oxygenase activity and protection, Experimental and Molecular Medicine, 38(4): 393-400 (2006). Curcumin therefore ameliorates damage to endothelial cells from exposure to gas bubbles from compressed gases in diving. Kunchandy, et al., Oxygen radical scavenging activity of curcumin. Int'l J. Pharmaceutics 58:237-240 (1990); Unnikrishnan, et al., Curcumin inhibits nitrogen dioxide induced oxidation of hemoglobin, Mol. Cell. Biochemistry 10: 35-37 (1995); Coyly et al., Effect of turmeric, turmeric and curcumin on H2O2-induced renal epithelial (LLC-PK1) cell injury, Free Radic. Biol. Med, 24:49-54 (1998); Circumin is also a powerful anti-oxidant. Cikrikci, et al., Biological activity of curcuminoids isolated from curcuma longa, Rec. Nat. Prod. 2(1):19-24 (2008). The curcumin formulation Meriva® by Indena produces more bioavailability for curcumin than pure forms. This formulation contains a ratio of 1:2:2 for Curcumin, Phosphatidylcholine, and Microcrystalline Cellulose. A preferred embodiment of the invention contains the Meriva® formulation for bioavailability of Curcumin in the amount of 150 mg/Liter of water, but an approximate ranger of 100-1000 mg of Meriva® formulation (or Curcumin contained therein in the range of 20-200 mg) would also be desirable and conform to the goals and function of the invention. In other embodiments of the invention, Curcumin not formulated as Meriva®, can be substituted. Because the bioavailability of non-Meriva® Curcumin is less, the range for this component should be increased approximately 20 fold so the preferred range on non-Meriva® Curcumin would be approximately 20 fold the Meriva® or 400-4000 mg.

N-Acetyl-Cysteine. Cysteine is one of the most ubiquitous anti-oxidants in the body and is an essential component of another powerful anti-oxidant, Glutathione. Glutathione is the most versatile and plentiful anti-oxidant in humans, and can be included in the invention in another embodiment. Research shows that doses of N-Acetyl-Cysteine (NAC) ameliorate the effects of ROS from exercise and tissue injury. Kerksick, et al., Anti-oxidant role of glutathione and n-acetyl-cysteine supplements and exercise-induced oxidative stress, J. International Soc. Sports Nutrition 2(2): 38-44 (2005); Guney, et al., N-acetylcysteine prevents vasospasm after subarachnoid hemorrhage, World Neurosurgery (2009). Studies have shown that NAC supplements increase cysteine and therefore glutathione levels in the body. NAC increases the levels of Gluthathione which in turn binds reactive oxygen species (ROS) and protects the divers from ROS toxicity. ROS toxicity is what causes seizures and black out underwater. NAC can increase bioavailability of Nitric Oxide (NO) which, as shown above, in turn has a protective effect against gas bubbles. Gal, et al., Tissue oxygenation and n-acetylcysteine in patients with diabetic foot ulcers, Undersea and Hperbaric Medical Society Journal (2006).

L-Arginine. Among other things, L-Arginine has the effect of increasing blood glucose and insulin in blood while decreasing free fatty acids in blood. Pu-Hsi Tsai, et al., Effects of arginine supplementation on post-exercise metabolic responses, Chinese Journal of Physiology 52(3): 136-142 (2009). Administration of a NO donor before a dive protects against bubble formation and death. Wisloff, et al., Exercise and nitric oxide prevent bubble formation: a novel approach to the prevention of decompression sickness?, J. Physiol. 555.3, pp 825-849 (2004). L-arginine also improves smooth muscle performance and improves urea cycle function and nitrogenous waste removal.

Ionized Electrolytes. Ionized electrolytes potassium, calcium, magnesium, and manganese make the blood more acidic and increase the O₂—CO₂ exchange performance. The ionized electrolytes increase acidity in the blood which increases the dissociation of oxygen from hemogloblin under the Bohr effect—so that more oxygen is released from hemoglobin into the mitochondria. Increasing acidity in the blood lowers pH for better dissociation of oxygen from hemoglobin to meet the demands of highly active tissues such as the muscles and brain. Poyart, et al., Measurement of the intrinsic alkaline Bohr effect of various human haemoglobins by isoelectric focusing, Biochem. J. 195:493 (1981); Fink, et al., Potassium and ionic strength effects on the isometric force of skinned twitch muscle fibres of the rat and toad, J. Physiol. 370: 317-37 (1986). The anti-oxidant NAC can bind copper and zinc so the invention adds it back. Plus, adding ionized electrolytes combats against formation of gas bubbles in blood vessels and fluid loss in diving. Gempp, et al., Preventive effect of pre-dive hydration on bubble formation in divers, Br. J. Sports Med. 43: 224-28 (2008).

Vitamins. Anti-oxidants such a Vitamin C and α-tocopherol (vitamin E) prevent increases of AST and ALT. Ikeda, et al., Supplementation of antioxidants prevents oxidative stress during a deep saturation dive, Tohoku J. Exp. Med. 203: 353-57 (2004).

Inositol. This is an organic osmolyte (which adds to osmolarity) important in keeping cells round, full, plump, and viable. The formula is C₆O₆H₁₂.

Citric Acid Substrates. These are added to provide substrate for running the Citric Acid Cycle, the energy production cycle ubiquitous in all cells: Fumaric Acid (C₄H₄O₄), Citric Acid (C₆H₈O₇), Folic Acid (C₁₉H₁₉H₇O₆), and Biotin (C₁₀H₁₆N₂O₃S).

What is claimed is:

1. A performance beverage in accordance with the disclosure herein.

2. A powder performance beverage mix in accordance with the disclosure herein.
